SEQUENCE LISTING (4 sequences)

SEQ ID NO: 1 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 2 - protein, 269 aa, Artificial seqence
  Fusion protein comprising EMP1, linker, and human IgG-like domains
QIQGGTYSCHFGPLTWVCKPQGGGSGGGSGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 247 aa, Artificial sequence
  Fusion protein comprising EMP1, linker and human IgG regions
QIQGGTYSCHFGPLTWVCKPQGGGSCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 484 aa, Homo sapiens
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

(19) United States
(12) Patent Application Publication
(10) Pub. No.: US 2010/0266591 A1
(43) Pub. Date: Oct. 21, 2010

(54) METHOD OF TREATING ERYTHROPOIETIN HYPORESPONSIVE ANEMIAS

(76) Inventors: Peter Bugelski, Radnor, PA (US); Renold Capocasale, Mt. Laurel, NJ (US); Dorie Makropoulos, Radnor, PA (US); Ram Achuthanandam, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/744,533

(22) PCT Filed: Nov. 24, 2008
(86) PCT No.: PCT/US08/84497

§ 371 Date: May 25, 2010

Related U.S. Application Data
(60) Provisional application No. 61/019,367, filed on Jan. 7, 2008.

(51) Int. Cl. A61K 39/395; A61P 7/06

(52) U.S. Cl. 424/134.1

(57) ABSTRACT

The invention relates to methods of using compositions comprising EPO-mimetic peptides to treat anemia. The invention relates to methods of treating disorders characterized by the insufficient amounts of erythrocytes and hemoglobulin in the blood due to myelodysplastic syndrome (MDS) or by hemoglobinopathies, such as alpha- or beta-thalessemia or sickle cell disease.

### PRIOR APPLICATION

This application claims priority to U.S. application No. 61/019,367, filed Jan. 7, 2008, which is entirely incorporated herein by reference.

### BACKGROUND OF THE INVENTION

1. Field of the Invention

The invention provides methods of treating anemias of genetic etiology and those secondary to chronic disease using EPO-mimetic peptide compositions. The invention comtemplates the treatment of anemia especially under conditions where the anemia is hyporesponsive to recombinant human erythropoietin.

2. Description of the Related Art

Anemia has multiple etiologies: it may be caused by dietary deficiencies, e.g. iron, or congenital abnormalities, or it may be associated with other pathologies, such as chronic kidney disease, cancer, or human immunodeficiency virus (HIV) infection. In turn, anemia is associated with an increase in morbidity and mortality in patients with end-stage renal disease, cancer, or HIV infection. Identifying the most appropriate treatment for each case of anemia requires an understanding of the etiology of the anemia and, if present, of the causative medical condition. Anemia accompanying chronic kidney disease is due diminished production of the natural erythropoeisis inducing hormone, erythropoietin. In other instances, such as megaloblastic anemia, insufficient erythropoiesis is due to vitamin or folate deficiency. Diverse presentations of anemia, require an equally diverse pharmacological and supportive treatment approaches.

Naturally occurring erythropoietin (EPO) is a glycoprotein hormone that is the principle growth factor mediating production of red blood cells (erythropoiesis). Currently approved products which act through stimulation of erythropoiesis include products comprising recombinant human erythropoietin (EPREX™, PROCRIT™, epotin-alfa, Neorecormon™, epotin-beta) and darbepoetin alfa (a recombinantly produced protein which is a hyperglycosylated variant of erythropoietin). These, termed erythropoiesis stimulating agents (ESAs), are approved for avoiding transfusion in anemia secondary to cancer chemotherapy and chronic kidney disease (CKD). Numerous other compositions for stimulating erythropoiesis are being explored. ESAs have been identified by screening peptide libraries for compositions that bind to and activate the erythropoietin receptor (EPO-R), e.g. EMP-1 and variants (Johnson et al., 1998 and Wrighton et al., 1996) and PEGylated synthetic peptide-derived constructs (Fan et al 2006, U.S. Pat. No. 6,703,480, U.S. Pat. No. 7,084,245).

In cancer chemotherapy and CKD, the rationale for administration of ESAs is replacement or supplementation of endogenous erythropoietin lost or present at insufficient levels to maintain or replenish mature erythrocytes. However, possibly over 50% of cancer chemotherapy patients fail to respond adequately to conventional doses of approved ESAs, erythropoietin up to 400,000 units weekly or darbepoietin of 200 microgram every two weeks (Vasu et al., 2006) and as many as 15% of CKD patients gain only limited benefit (Rossert et al., 2007). Approved ESAs have also been used “off-label” in the hemoglobinopathies, e.g., beta-thalassemia (Makis et al., 2001 and Kohli-Kumar et al., 2002), sickle cell anemia (Rodgers et al., 1993) and in myelodysplastic syndrome (MDS) (Mundle et al., 2006 and Musto et al., 2006). In these conditions, anemia results from defective red cell production or shortened red blood cell life span and approved ESAs have had limited therapeutic success. Thus, there is a need for an ESA that will provide a more predictable response and provide therapeutic benefit in anemias that are resistant or hyporesponsive to EPO or EPO-derived ESAs.

### SUMMARY OF THE INVENTION

A method for treating a subject having a disorder characterized by a low blood hemoglobin level or a low level of red blood cells in the blood characterized as anemia caused by a hemoglobinopathy or myelodysplasia, which method comprises contacting the hematopoietic tissue of the patient with a therapeutically effective amount of the compound comprising dimeric polypeptides in which each polypeptide comprises an erythropoietin mimetic peptide (EMP) and a human immunoglobulin domain, wherein the dimeric polypeptide composition is capable of causing erythropoietin-dependent cells to proliferate. In specific embodiments of the invention, the hemoglobinopathy is caused by the subject has sickle cell disease and expresses HgbS or the subject has beta-thalassemia. In another embodiment, the patient is suffering from a chronic disease of the kidney causing myelodysplasia leading to anemia. In yet another embodiment, the patient has a defect in a hematopoietic tissue cell stem factor receptor causing myelodysplasia leading to anemia.

In one embodiment of the method of treating anemia in a subject the hematopoietic tissue is contacted in vivo and the composition of the invention is administered to the subject. In another embodiment of the method of treating anemia the hematopoietic tissue is contacted with the composition of the invention ex vivo.

In one embodiment of the invention, the EMP is designated EMP-1. In a specific embodiment of the invention, the composition comprises a homodimer of disulfide linked polypeptides of SEQ ID NO: 2 or SEQ ID NO: 3.

### BRIEF DESCRIPTION OF THE DRAWING

FIG. 1 is a graph showing the effect of EPO on Hgb in C57/Bl vs. Tg197 mice, where expression of human tumor necrosis factor-α models anemia due to chronic inflammatory disease.

FIG. 2 is a graph showing the effect of CNTO 530 as compared to epoetin-α and darbepoetin alfa (darbe) on serum Hgb in Tg197 mice following a single s.c. administration of equivalent UT7 activity units.

FIG. 3 is a graph showing the effect of epoetin-α on Hgb in c-kit deficient (W/Wv) mice which is model of SCF receptor deficiency.

FIG. 4 is a graph showing the effect of CNTO 530 as compared to epoetin-α on hemoglobin in c-kit deficient (W/Wv) mice.

FIG. 5 is a graph showing the effect of CNTO 530, epoetin-α and darbepoetin doses (expressed as UT-7 units/kg) on Hgb in Th3+/C57BL/6 mice, a model of beta-thalassemia.

FIG. 6 is a graph showing the effect of CNTO 530 (0.3 mg/kg) on HbF in human HbS transgenic mice as measure by ion exchange chromatography, where the peak fraction (4) was used to assess the pre-/post-dosing ratio.

FIG. 7 shows a stained acid agarose electrophoretic gel loaded with red blood cell lysate from a representative in human HbS transgenic mouse pre- and nine days post-treatment with CNTO530 (0.3 mg/kg) and a third line loaded with Hgb standards; F=HbF, A=HbA, S=HbS, C=HbC.

### BRIEF DESCRIPTION OF THE SEQUENCE LISTING

| SEQ ID NO: | Description | Reference |
|---|---|---|
| 1 | Mature human erythropoietin | P01588 |
| 2 | Dimeric EMP1 construct: CNTO 528 | |
| 3 | Dimeric EMP1 construct: CNTO 530 | |
| 4 | Human erythropoietin receptor | NP_000112 |

### DETAILED DESCRIPTION OF THE INVENTION

### Abbreviations

EMP erythropoietin mimetic peptide; EPO human erythropoietin; EPO-R erythropoietin receptor; ESA erythropoiesis stimulating agents; Hgb hemoglobin; Hct hematocrit; HPFH hereditary persistence of fetal hemoglobin; SCF stem cell factor; IL interleukin; GH growth hormone; GM-CSF granulocyte macrophage colony stimulating factor; MCH mean (red) cell hemoglobin; MCV mean (red) cell volume; RBC red blood cells, erythrocytes; TNF-α tumor necrosis factor alpha;

### DEFINITIONS

By “anemia” is generally meant a hemoglobin level in the blood which is below normal values and is associated with consequences to the health and performance of an individual which include weakness, dizziness, shortness of breath and risks of more severe morbidities. Low serum Hgb can be as a result of less than the normal number of red blood cells or less Hgb than normal in the RBC causing them to be too small (microcytic, low MCV) or underpigmented (hypochromic, low MCH). Normally formed RBC in low numbers will cause blood hematocrit (Hct, percentage of blood volume occupied by RBC) to fall. Thus, the expression of serum Hgb levels encompasses all possible scenarios of erythrocyte number and erythrocyte Hgb complement and is usually given in gm Hgb/dL blood. The definition of normal Hgb in adult humans varies but average Hgb for adult males is between 13-14 gm/dL and for females between 11.6-12.3 gm/dL where values below these lower level can be considered anemia (Beutler and Waalen 2006 Blood 107:1747-1750). Other factors such as age, genetic background, and elevation at which an individual resides may affect the amount of serum Hgb required for good health and performance. Responsiveness to anemia therapy is measured as the increase in serum Hgb concentration.

By “EPO” or “erythropoietin” or “rhEPO” is meant a composition which is a polypeptide chain monomer synthesized in the human body or made recombinantly having the 166 amino acid sequence as shown in SEQ ID NO: 1, the identical amino acid sequence of isolated natural erythropoietin, and in the Uniprot accession No. P01588 mature chain. EPO may include active cleavage products, especially C-terminal truncations, be glycosylated or non-glycosylated, or be otherwise modified such as by PEGylation, or carbamoylation (see WO2004003176) at specific or non-specific sites on the polypeptide chain. In the body, EPO is made primarily in the kidney and to a lesser extent, in the liver. Recombinantly made human EPO and recombinant modified EPO compositions have been shown to display bioactivities other than erythropoiesis (Bunn, H F 2007. Blood 109: 868-873).

By “EPO-derived” ESA is meant a composition which is a polypeptide chain monomer capable of being made recombinantly which has substantial sequence identity with EPO. By substantial sequence identity is meant that, using an sequence alignment algorithm, the sequence of the EPO-derived ESA and EPO can be matched and the percent identity between the two sequences is greater than 80%. Examples of EPO-derived products include darbepoetin alfa (ARANESP™, Amgen, Calif.) which comprises a variant polypeptide chain sequence of SEQ ID NO: 1 (EPO) as described in U.S. Pat. No. 7,217,689 and C.E.R.A. (Continuous erythropoiesis receptor activator) also known by its chemical name, methoxypolyethylene glycol-epoetin beta, (MICERA, Roche, Switzerland) is a ESA whose structure incorporates a large polymer chain providing it an extended half-life, and others (EP1196443B1).

By “EPO-mimetic peptide” is meant a composition having natural, or a combination of natural and non-natural amino acid residues connected in sequence whereby substantially none of the sequence can be aligned with naturally occurring EPO but where the EPO-mimetic peptide exhibits erythropoietic activity which is similar to EPO, such as but not limited to EPO-R specific binding and stimulation of UT7 cell proliferation (Komatsu, N., et al. Blood 82(2), 456-464, 1993). An example of an EPO-mimetic peptide is given by the sequence GGTYSCHFGPLTWVCKPQGG (residues 4-23 of SEQ ID NO: 2 and 3).

The human EPO receptor or “EPO-R” has an amino acid sequence given by NCBI accession No. NP_{—}000112 (SEQ ID NO: 4) where the mature chain is represented by residues 25-508 and has and extracellular domain, transmembrane domain, and intracellular domain.

By “erythropoiesis” also “erythrocytopoiesis” is meant the process whereby multipotent hematopoietic stem cells (HSC) differentiate to the mature red blood cells (erythrocytes) which are anucleated cells comprised principally of mature hemoglobin tetramers. Developing erythroid cells respond to signals from stromal cells of the bone marrow or spleen. The process of erythropoiesis takes place in the bone marrow where erythroblasts are organized into erythroblastic islands that consist of macrophages surrounded by developing erythroid precursors. Macrophages provide many of the cellular mediators that control erythropoietic activity: GM-CSF, IL-3, and stem cell factor (SCF) generate colony-forming unit erythroid macrophage-granulocyte megakaryocyte (CFU-GEMM) and burst-forming unit erythroid (BFU-E), whereas TGF-β, TNF-alpha (Dufour et al. 2003 Blood 102:2053-2059), and MIP1-alpha inhibit cell cycle activity and BFU-E development. EPO induces the expansion of colony-forming unit erythroid (CFU-E) cells and initiates differentiation through a number of erythroid-specific events, a process that generally proceeds over a two day period. SCF and EPO synergize to drive the proliferation of human erythroid progenitors and precursors.

### Compositions

CNTO 528 and CNTO 530 are EPO-mimetic peptide antibody fusion proteins, which in their mature form include two copies of the EMP1 peptide and portions of a human IgG antibody (U.S. Pat. No. 7,241,733; US Ser No. 2004935005; WO2004002424 A2; WO2005032460). CNTO528 is a homodimer of polypeptide shown in SEQ ID NO: 2 and CNTO 530 is a homodimer of SEQ ID NO: 3 both covalently joined by disulfide bonds via cysteine residues present in the immunoglobulin derived portion of the molecule, and which may or may not be glycosylated. Other dimeric peptide-derived constructs such as those described in U.S. Pat. No. 6,703,480 and U.S. Pat. No. 7,084,245 may also be useful in the methods of the invention.

CNTO 528, CNTO 530 and HEMATIDE are pharmacologically active in a variety of in vitro and in vivo test systems. While monomeric EMP-1 has a binding affinity for EPO-R of about 200 nM, CNTO 528 and CNTO 530 bind the EPO-receptor with a binding constant of approximately 10 nM and are active in suppressing apoptosis and stimulating cell growth in a variety of in vitro models of erythropoiesis and are stimulate erythropoiesis in animal models in vivo. It has been recognized that the dimeric forms of EPO-R binding peptides, peptide-derivatives, and other EPO-mimetics can be more potent than monomers in activating the EPO-R (Livnah, O. et al. 1996. Science 273: 464-471; Johnson et al. 1997. Chem Biol 4:939-50) as dual binding domains, when properly oriented (Balinger and Wells. 1998. Nat Structural Biol 5: 938-940) bring the extracellular domains of the EPO-R in situ to the proper proximity and orientation to form a signaling complex. Thus, ESAs which are not substantially identical to the amino acid sequence of human EPO, present in dual or dimeric form, such as but not limited to those comprising dimeric forms of SEQ ID NO: 2 and 3, are subject compositions of the invention.

The dimeric forms of SEQ ID NO: 2 and 3, further comprise a structure known to resemble the crystallizable fragment resulting from papain cleavage of an G-class immunoglobulin (Fc). The Fc region of an antibody provides certain non-antigen binding functions such as the ability to bind and interact with complement, the ability to bind and activate Fc-specific receptors on circulating and non-circulating cells and tissues of the immune system. The Fc region of the composition also imparts an advantage related to the ability to remain in the plasma compartment of the bloodstream and resist renal filtration or be transported across cell membranes by the receptors known and unknown including the FcRn receptor. These and other advantages imparted by the complex structures of the dimeric forms of SEQ ID NO: 2 and 3 will be recognized by those practitioners of art of antibody engineering. Thus, the dimeric presentation of EMP-peptides that has been described previously combined with the Fc-region properties of the mature structure are uniquely suited to the practice of the methods of erythropoietic stimulation as demonstrated by the dimeric forms of SEQ ID NO: 2 and 3.

### Methods of Testing and Dosing the Erythropoietic Activity of the Compositions

An “international unit” or “IU” of EPO activity is defined as the amount of EPO (SEQ ID NO: 1) giving the same amount of erythroid stimulus as 5 microgram of cobalt. Cobalt, a naturally-occurring element with properties similar to those of iron and nickel, induces a marked and stable polycythemic response through a more efficient transcription of the erythropoietin gene. The international reference standard for EPO assays use isolated human urinary EPO. EPO standards are calibrated against reference EPO preparations, in particular, the Second International Standard for Recombinant-Derived EPO supplied by the World Health Organization (WHO) or the National Institute for Biological Standards and Control (NIBSC). Units of activity are defined as the amount of EPO that gives the same amount of erythroid stimulation as 5 micromoles of cobalt. However, usually EPO preparations are calibrated in bioassays against a reference standard. Human urinary EPO typically has a specific activity of about 70,000 U/mg of protein while values reported for human recombinant EPO may range between 100,000 to 200,000 U/mg depending on the carbohydrate (glycosylation) content of the product.

Other in vivo and in vitro assays can be used to assess the amount of erythropoietic activity. For example, erythropoietic activity can be measured in vitro in the short term culture of cell lines of hematopoietic lineage, e.g. bone marrow or spleen derived cells (FDC-P1/ER, a well characterized nontransformed murine bone marrow derived cell line in which EPO-R has been stably transfected (Dexter, et al., 1980 J. Exp. Med. 152:1036-1047), or EPO responsive tumor cell lines such as TF1 (Kitamura, et al., 1989 Blood 73:375-380) or UT7 cells (Kitamura et al. 1989. J Cell Physiol. 140:323; Komatsu, N., et al. Blood 82(2), 456-464, 1993), or cell lines engineered to be dependent upon EPO for growth.

Particularly useful in identifying and calibrating compositions useful in the method of the invention is the UT7 cell proliferation assay. The UT7 is a human leukemic cell line that has been adapted to become EPO-dependant. In order to use the UT7 cell proliferation assay for selection of a composition or a dose to be administered to a subject having low blood hemoglobin content, the cells are washed free or normal culture medium and starved for EPO for 24 hours prior to assay. For example, the UT-7 cell starvation can proceed in IMDM media with added L-glutamine and FBS at 5% (I5Q). The cells are then prepared and suspended in the appropriate media to a final concentration of 6×10⁵ cells/mL (yields a final concentration of 30,000 cells per 96-well chamber). An EPO standard is prepared by diluting EPO stock to 5 ng/mL followed by 1:2 serial dilutions down to a concentration of 0.0098 ng/mL in I5Q media. The resulting dilutions provides standards at concentrations of 2.5 ng/mL to 0.0024 ng/mL (after a final r-fold dilution within the test well). The test sample is diluted in a similar manner. A 50 microL aliquot of the UT-7 cell suspension is transferred to the corresponding wells and the plates were incubated at 37° C. for 48 hours. Cell proliferation is assessed using a vital stain such as Promega's MTS solution (Promega, Madison, Wis.) according to the manufacturer's instructions. The EC50 is calculated from a curve fit of concentration vs proliferation as measured by the increase in absorbance of the chromophore or other signal. The EC50 for unmodified EPO is approximately 1.8×10⁻¹¹ M. Using this value, UT7 units for other agents can be standardized to EPO. To calculate UT-7 rHuEPO equivalents:

UT-7 Units/ug=Mol wt×C/EC₅₀ for the test compound, where C is a constant derived from the activity of rHu EPO under the same assay conditions, and a known pharmacological specific activity of rHu EPO is known (e.g. 120 U/ug):

$$C = \frac{120 U / μg \times {EC}_{50} for rHuEPO}{34 kD} = 33.7$$

The EC₅₀ for test compound is derived from a curve fitted to concentration vs response using the UT-7 viability assay, and finding the concentration at which 50% maximal cell proliferation activity is achieved. Thus, the amount or dose of an ESA to be administered may be converted from mg/kg to UT-7 U/kg by multiplying the respective mg/kg dose by the in vitro activity of each compound.

While erythropoiesis can be recapitulated in vitro and studies with BFU-e and CFU-e in semi-solid cell cultures have added to our understanding of this process, in vivo, bone marrow stromal cells and macrophages play an important role in creating microenvironments for stem cells and erythroblastic islands, respectively (Sadahira and Mori, 1999). These cells express a variety of cytokines and adhesion molecules, and macrophages are postulated to act as nurse cells for erythroblasts. Since bone marrow macrophages usually contain substantial amounts of ferritin, it is likely that they also have an influence on iron metabolism. Depending on the preparation and culture techniques, the function of these cells and their cytokines may be lost in in vitro systems. Thus, observations made in vitro may not translate directly to an in vivo setting.

In vivo bioassays for erythropoietic activity may be further influenced by other compounds and endogenous substances that modify erythropoeisis. For these reasons, in vivo assays using animal must be carefully controlled. Models of disease which reflect these inherent differences and control parameters such as dietary iron intake, presence or absence of inflammation, other growth factors, steroid hormones, etc. can be used to study aspects of erythropoiesis and response to therapy.

Doses of the EPO-mimetic fusion proteins exemplified by the homodimer structures of SEQ ID NO: 2 and 3 and as described herein, may be administered as equivalent in activity to EPO which can be used from 0.1 units/ml to 20 units/ml, preferably from 0.5 units/ml to 2 units/ml, or any range or value therein. In other applications of the use of EPO-mimetic fusion proteins which are either erythropoitic or non-erythropoeitic the dose administered need not be related to erythropoietic units.

Applicants have unexpectedly discovered, using animal models of hemoglobinopathies and myelodysplasia, that the non-erythropoietin derived ESAs comprising dimeric constructs of EPO-mimetic peptides may be used to advantageously to treat these diseases. In addition, applicants have shown, using in vivo models, that CNTO528 and CNTO530 stimulate erythropoiesis and hemtopoeisis evidenced as an increase in blood Hgb which, when compared to other ESAs in the same model, was to a greater extent and/or for a more sustained duration based on in vitro EPO-dependent cell proliferation activity.

### Methods of Using the Compositions

Approximately 5-10% of patients with chronic kidney disease demonstrate hyporesponsiveness to ESA, defined as a continued need for greater than 300 IU/kg per week erythropoietin or 1.5 μg/kg per week darbepoetin administered by the subcutaneous route. Such hyporesponsiveness contributes significantly to morbidity, mortality and health-care economic burden in chronic kidney disease and represents an important diagnostic and management challenge. The commonest causes of ESA resistance are non-compliance, absolute or functional iron deficiency and inflammation. It is widely accepted that maintaining adequate iron stores is important for reducing the requirements for, and enhancing the efficacy of ESA. ESA hyporesponsiveness may be due to various factors specific to the ESA composition or to host factors. Some well-established causes of ESA hyporesponsiveness include inadequate dialysis, hyperparathyroidism, nutrient deficiencies (vitamin B12, folate, vitamin C, carnitine), angiotensin-converting enzyme inhibitors, angiotensin receptor blockers, aluminium overload, antibody-mediated pure red cell aplasia, primary bone marrow disorders, myelosuppressive agents, haemoglobinopathies, haemolysis and hypersplenism (see Johnson, D W et al. (2007) Erythropoiesis-stimulating agent hyporesponsiveness. Nephrology 12 (4), 321-330 for a review).

While not wishing to be bound by any one theory of operation, certain mechanistic considerations define differences between EPO-mimetic peptide compositions and single chain polypeptide compositions. The functional mimicry of the hematopoietic growth hormone, erythropoietin (EPO), can be achieved by certain dimeric presentations of the EMP-1 peptide. The crystal structure at 2.8 angstrom resolution of a complex of this agonist peptide with the extracellular domain of EPO receptor reveals that an EMP-1 peptide dimer induces a dimerization of the receptor. While the EPO-R and human growth hormone (hGH) receptor share certain structural aspects, the hGH receptor-ligand complex differs from the EPO-EPOR dimer complex and suggests that more than one mode of dimerization may be able to induce signal transduction and cell proliferation (Livnah, et al. 1996 Science 273(5274): 464-471). CNTO528 and CNTO530, which represent homodimers of EMP-1 fused to linking regions and to immunoglobulin G class constant domains such as but not limited to SEQ ID NO: 2 and 3, achieve the spatial orientation to induce EPO receptor signaling (SEQ ID NO: 4) and stimulate erythropoiesis (See WO04/002424; Bugelski et al. (2005) Blood 106 (11): Abstract 4261; Franson et al. (2005) Blood 106 (11): Abstract 4283; WO05032460A2), however, due to the unique nature of these constructs and resulting homodimer 3-dimensional conformations, unique aspects of their ability to interact with EPO receptor(s) may provide these molecules with an activity spectrum which differs from natural EPO. In addition, as the primary, secondary, tertiary, and quaternary structures of peptide-mimetic ESAs, including CNTO530 and CNTO528, are unlike natural EPO, the constructs will have different patterns of distribution, metabolism, and antigenicity or lack thereof. Applicants have unexpectedly found, using animal models of human anemia resulting from hemoglobinopathies and myelodysplasia, that EMP1-comprising constructs provoke enhanced erythropoiesis in terms of extent and/or duration of hemoglobin response as compared to a comparable level of in vitro based bioactivity units (ESA-dependent cell proliferation) of the single polypeptide chain of recombinant human EPO or a single polypeptide chain variant of the natural EPO protein sequence (darbepoetin).

### Hemoglobinopathies

The human hemoglobins are encoded in two tightly linked gene clusters; the alpha-like globin genes on chromosome 16, and the beta-like genes on chromosome 11. Important regulatory sequences flank each gene and promoter elements are upstream. Sequences in the 5′ flanking region of the gamma and the beta genes appear to be crucial for the correct developmental regulation of these genes, while elements that function like classic enhancers and silencers are in the 3′ flanking regions. The locus control region (LCR) elements located far upstream appear to control the overall level of expression of each cluster. These elements achieve their regulatory effects by interacting with trans-acting transcription factors. The latter also appear to modulate genes specifically expressed during erythropoiesis, such as the genes that encode the enzymes for heme biosynthesis. Normal red blood cell (RBC) differentiation requires the coordinated expression of the globin genes with the genes responsible for heme and iron metabolism.

There are five major classes of hemoglobinopathies: structural (e.g. sickle cell), variants (e.g. with altered O₂ affinity), thalassemias (altered or miscoordinated hemoglobin chain synthesis), hereditary persistence of fetal hemoglobin (HPFH), and acquired (e.g. methemoblobin). Thalassemic hemoglobin variants combine features of thalassemia (e.g., abnormal globin biosynthesis) and of structural hemoglobinopathies (e.g., an abnormal amino acid sequence).

The sickle cell syndromes are caused by a point mutation in the beta-globin gene that changes the sixth amino acid from glutamic acid to valine and designated hemoglobin S (HgbS). HgbS polymerizes reversibly when deoxygenated causing stiffening of the erythrocyte membrane and the characteristic sickled shape. Sickled erythrocytes are adhesive and inflexible, adhering to each other and vascular endothelium. These abnormalities provoke unpredictable episodes of microvascular vasoocclusion and premature RBC destruction both by frank hemolysis and due to removal by the spleen. Prominent manifestations include episodes of ischemic pain (i.e., painful crises) and ischemic malfunction or frank infarction in the spleen, central nervous system, bones, liver, kidneys, and lungs.

The thalassemia syndromes are inherited disorders of alpha- or beta-globin biosynthesis. Mutations causing thalassemia can affect any step in the pathway of globin gene expression: transcription, processing of the mRNA precursor, translation, and posttranslational metabolism of the -globin polypeptide chain. The most common forms arise from mutations that derange splicing of the mRNA precursor or prematurely terminate translation of the mRNA. Unbalanced accumulation of globin subunit occurs because the synthesis of the unaffected globin proceeds at a normal rate. The reduced production of complete hemoglobin tetramers (alpha₂beta₂) results in erythrocyte hypochromia and microcytosis. Clinical severity varies widely, depending on the degree to which the synthesis of the affected globin is impaired, altered synthesis of other globin chains, and coinheritance of other abnormal globin alleles. Both beta-gene derived and alpha-gene derived, alpha- and beta-thalassemias, are known and characterized. The most common form of thalassemia is beta-thalassema major, also called Cooley anemia, caused by over 200 mutations leading to altered production of the beta-chain of hemoglobulin. Other forms include, but are not limited to, beta-thalassema minor, and beta-thalassema intermedia.

HPFH is characterized by continued synthesis of high levels of HgbF, fetal hemoglobin, in adult life. No deleterious effects are apparent, even when all of the hemoglobin produced is HgbF. Thus, any stimulus which would promoted HgbF formation in patients carrying genetic defects in the alpha- or beta-genes are their processing, such as in sickle cell anemia and thalassemia, could prove to be efficacious.

### Bone Marrow Failure

Myelodysplasia, myelodysplastic syndrome (MDS), aplastic anemia, pure red cell aplasia (PRCA), and myelophthisis are diseases characterized by bone marrow failure. The myelodysplastic syndromes (MDS, formerly known as “preleukemia”) are a diverse collection of hematological conditions characterized by ineffective production of blood cells and varying risks of transformation to acute myelogenous leukemia. MDS is classified within the haematological neoplasms. Anemia requiring chronic blood transfusion is frequently present. The hypoproliferative anemias are normochromic, normocytic or macrocytic and are characterized by a low reticulocyte count. Deficient production of RBCs occurs with marrow damage and dysfunction, which may be secondary to infection, inflammation, and cancer. Anemia in these disorders is often not a solitary or even the major hematologic finding. The bone marrow failure of MDS may result in pancytopenia: anemia, leukopenia, and thrombocytopenia.

Haematopoiesis (sometimes also haemopoiesis or hemopoiesis) is the formation of blood cellular components. All of the cellular components of the blood are derived from haematopoietic stem cells. Glycoprotein growth factors are known to regulate the proliferation and maturation of the cells that enter the blood from the marrow, and cause cells in one or more committed cell lines to proliferate and mature. A common myeloid progenitor cell, pluripotent stem cell, responds to growth factors including SCF, IL-3, GM-CSF, and EPO to produce erythroid cells and erythrocytes, a process called erythropoiesis. Erythropoiesis is highly dependent upon and regulated EPO which is produced in the kidneys in response to hypoxia. However, EPO receptors are found on other cells types in addition to myeloid progenitor cells and, as previously noted, a variety of downstream signaling events result from EPO receptor activation by ligands. Non-erythropoietic related cardiac and neural tissue protection by certain derivatives erythropoietin derivatives, lysine carbamylated erythropoietin, where erythropoietic activity is abolished have also been noted (Leist et al. 2004 Science 305: 239).

### Therapeutic Applications

The present invention provides a method for modulating or treating anemia, in a cell, tissue, organ, animal, or patient including, but not limited to, at least one of any anemia; pediatric and/or adult cancer-associated anemia; cancer treatment related anemia; radiotherapy or chemotherapy related anemia; parasite, viral or bacterial infection related anemia; anemia due to renal damage or failure; anemia of prematurity, anemia due to hemoglobinopathies, or anemia due to bone marrow failure. More specifically, the anemia may be associated with primary or secondary effects due to cancer or infections including lymphoma, myeloma, multiple myeloma, AIDS; end-stage renal disease (ESRD), anemia associated with dialysis, chronic renal insufficiency; hemopoietic diseases, such as congenital hypoplastic anemia, Fanconi's anemia; thalassemias including but not limited to beta-thalassemia and alpha-thalassemia, and sickle cell disease.

The ESA compositions of the present invention can also be used for non-renal forms of anemia induced, for example, by chronic infections, inflammatory processes, radiation therapy, and cytostatic drug treatment; or be encompassed by myelodysplastic syndrome (MDS) and other conditions in which chronic illness suppresses bone marrow and erythropoiesis.

The present invention also provides a method for modulating or treating a patient exhibiting anemia related to infectious disease in a cell, tissue, organ, animal or patient, including, but not limited to, at least one of acute or chronic bacterial infection, acute and chronic parasitic or infectious processes, including bacterial, viral and fungal infections, HIV infection/HIV neuropathy, meningitis, hepatitis, septic arthritis, peritonitis, pneumonia, epiglottitis, E. coli 0157:h7, hemolytic uremic syndrome, thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, vital-associated hemaphagocytic syndrome, vital encephalitis/aseptic meningitis, and the like.

The present invention also provides a method for modulating or treating a patient exhibiting anemia related the presence of cancer in a cell, tissue, organ, including, but not limited to, leukemia, acute leukemia, acute lymphoblastic leukemia (ALL), B-cell, T-cell or B-cell lymphoma, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, and the like.

The present invention also provides a method for modulating or treating a patient exhibiting anemia related a neurodegenerative disease in a cell, tissue, organ, including, but not limited to: multiple sclerosis, migraine headache, AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi. system disorder); demyelinating core disorders, such as multiple sclerosis, acute transverse myelitis; and disorders of the motor unit such as neurogenic muscular atrophies (anterior hom cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wemicke-Korsakoff syndrome; chronic alcoholism; Creutzfeldt-Jakob disease; Subacute sclerosing panencephalitis, Hallerrorden-Spatz disease; and Dementia pugilistica, and the like.

The present invention also provides a method for modulating or treating a patient exhibiting anemia related a cardiovascular disease, including, but not limited to, cardiac stun syndrome, myocardial infarction, congestive heart failure, stroke, ischemic stroke, hemorrhage, arteriosclerosis, atherosclerosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), chaotic or multifocal atrial tachycardia, regular narrow QRS tachycardia, specific arrythmias, ventricular fibrillation, His bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occulsion of the abdominal aorta and its branches, peripheral vascular disorders, occulsive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like.

Such a method can optionally comprise administering an effective amount of at least one composition or pharmaceutical composition comprising at least one ESA composition such as but not limited to the CH1-deleted EMP-1 peptide immunoglobulin fusion protein of the invention, including but not limited to SEQ ID NO: 2 or 3 or specified portion or variant to a cell, tissue, organ, animal or patient in need of such modulation, treatment or therapy. CNTO 528 (a homodimer of SEQ ID NO: 2) has been shown in a randomized, single-blind and placebo (PBO)-controlled study of 44 subjects in 5 dose cohorts (Stage 1, 35 subjects received a single IV administration of 0.03, 0.09, 0.3, 0.9 mg/kg CNTO 528 or PBO; Stage 2, 9 subjects received fractionated IV administrations of CNTO 528 or PBO on Days 1, 3 and 5 (3 infusions of 0.09 mg/kg or PBO); to be well tolerated and resulted in prolonged, dose-dependent erythropoietic responses with notably low inter-subject variability. The pharmacokinetics of IV CNTO 528 was linear and approximately dose proportional. Hemoglobin (Hgb) concentration increased in a dose dependent manner with a maximum effect occurring at day 22. Mean Hgb concentration remained 0.4 g/dL above baseline values at the last measurement, approximately 2.5 months after a single dose administration. A dose dependent increase in RBC count was observed with all RBC indices (MCV, MCH, MCHC) within normal range, indicating an increase in normocytic, normochromic RBCs. In all CNTO 528 treated subjects, a dose-dependent increase in soluble transferrin receptor concentration was observed. A dose-dependent increase in endogenous EPO concentration was observed, followed by a dose dependent decrease in endogenous EPO concentration. No immunogenicity was observed. This data provides proof of concept in humans for erythropoietic responses and up-regulation of endogenous EPO levels by an erythropoietic mimetic antibody fusion protein (Franson et al. 2005) Blood 106 (11): Abstract 4283).

The EPO-mimetic peptide comprising compositions can also be used ex vivo, such as in autologous marrow culture. The treated marrow is then returned to the patient, optionally after the patient has been treated with another agent or modality such as ionizing radiation. EPO-mimetic peptide comprising compositions, and, optionally other stem cell proliferation and differentiation factors, can also be used for the ex vivo expansion of marrow or peripheral blood progenitor (PBPC) cells. Optionally, the EPO-mimetic peptide comprising compositions can be used in combination with one or more other cytokines, including but not limited to SCF, G-CSF, IL-3, GM-CSF, IL-6 or IL-11, to cause the cells to differentiate and proliferate into high-density cultures, which are optionally then be returned to the patient.

While having described the invention in general terms, the embodiments of the invention will be further disclosed in the following examples.

### Background Non-Clinical Pharmacology for Compositions

In vitro, CNTO 528 was approximately 10-fold less potent on a molar basis than recombinant human EPO (rhEPO) in stimulating the growth of UT-7EPO cells. Despite the lower in vitro potency, when compared to rhEPO and darbepoetin in normal rats, a single subcutaneous dose of CNTO 528 caused a longer-lived reticulocytosis and a longer-lived increase in hemoglobin. As measured by flow cytometric methods CNTO 528 caused only minor changes in red cell distribution width (RDW) or mean cell volume (MCV), led to the release of mature reticulocytes and had no effect on mean platelet volume (MPV). CNTO 528 was shown to be efficacious in rat models of anemia and in a rat model of pure red cell aplasia (Bugelski et al. (2005) Blood 106: (11) Abstract 4261).

Using UT-7 cells, which display EPO-R and require an ESA for proliferation, competitive binding for the human EPO-R between CNTO 530 inhibited and ¹²⁵I-EPO was measured. CNTO 530 prevented EPO (0.5 nM) from binding to cells with an IC50 of about 60 nM. CNTO 530 was approximately 24-fold less potent than rHuEPO on a molar basis in a simple proliferation assay using UT-7 cells and CNTO 530 rescued cells deprived of EPO from apoptosis (EC50 for CNTO 530 was approximately 21 pM) and at higher concentrations, CNTO 530 caused a robust induction of proliferation (EC50 for CNTO 530 is approximately 55 pM).

In a human bone marrow colony formation assay was used to determine its effects on erythroid progenitor cell growth and differentiation. CNTO 530 induced a concentration-dependent increase in erythroid colony formation.

In vivo nonclinical pharmacology studies in normal animals, demonstrated that CNTO 530 caused a dose responsive stimulation of erythropoiesis in normal female C57Bl/6 mice when administered sub cut. at 0, 0.01, 0.3, 0.1 or 0.3 mg/kg. Basophilic erythroblasts were the most sensitive cells in the marrow and the no effect dose for CNTO 530 on basophilic erythroblasts was 0.01 mg/kg. There was no effect on non-erythroid cells in the marrow. Compared to recombinant human erythropoietin and darbepoetin, CNTO 530 was more effective and had a longer lasting effect on erythropoiesis. Using a single of CNTO530 administered to normal female Sprague-Dawley rats, sub cu. At 0, 0.01, 0.03, 0.1, 0.3 or 1 mg/kg; CNTO 530 caused a rapid dose-dependent, transient increase in reticulocytes and a sustained increase RBC, Hct and Hgb. The no effect dose for CNTO 530 for increasing reticulocytes was <0.01 mg/kg. For increasing Hgb, the no effect dose was 0.01 mg/kg and the ED50 was 0.1 mg/kg. CNTO 530 also caused a transient, non-dose-responsive (up to 1.5 fold) increase in white blood cell counts (WBC) The no effect dose for increasing WBC and was 0.01 mg/kg. At a dose of 1 mg/kg, CNTO 530 caused a transient (up to 1.5 fold) increase in mean platelet volume (MPV). The no effect dose for increasing MPV was 0.3 mg/kg. When given a high IV and SC dose of CNTO530 (0, 0.3, 3 and 30 mg/kg), female Sprague-Dawley rats exhibited a transient, non-dose-responsive increase in reticulocytes and a long-lived increase in red blood cell count (RBC), hemoglobin (Hgb) and hematocrit (Hct); transient, non-dose responsive increase in WBC; a transient, dose responsive increase in platelet counts and mean platelet volume (MPV).

In normal female rabbits, subcutaneous CNTO 530 at 0, 0.3, 3 or 30 mg/kg caused a transient, non-dose-responsive increase in reticulocytes and a longer-lived increase in RBC, Hct and Hgb. CNTO 530 caused an increase in mean platelet volume (MPV). The effect on MPV was dose-responsive between 0.3 and 3 mg/kg while a dose of 30 mg/kg had a similar effect as 3 mg/kg.

In Male and Female Cynomolgus Monkeys, CNTO530 given I.V. at 0, 0.03, 0.1, 0.3, or 3.0 mg/kg caused a dose-dependent increase reticulocyte counts, RBC, Hct and Hgb. The no effect dose was 0.1 mg/kg. High dose I.V. and sub cu. CNTO 530 in normal male cynomolgus monkeys (IV 0, 3 and 30 mg/kg) caused a transient, dose responsive increase in reticulocyte counts and a long-lived, non-dose responsive increase in red blood cell count (RBC), hemoglobin (Hgb) and hematocrit (Hct); transient, non-dose responsive increase in platelet counts and a long-lived, non-dose responsive increase in mean platelet volume (MPV).

### Example 1

### Model of Chronic Disease Related to Myelodysplasia

In this model, Tg197 mice which carry a human TNFalpha transgene with its 3′-untranslated region replaced by a sequence from the 3′-untranslated region of the beta-globin gene on a C57Bl/6 background, exhibit deregulated human TNFa gene expression. The pharmacodynamics of epoetin-α in C57Bl/6 and Tg197 mice was first compared. Secondly, CNTO 530, epoetin-α and darbepoetin in Tg197 mice was compared.

Materials and Methods. Nine-week old heterozygous Tg197-CBA F1 transgenic mice, age-matched C57Bl/6 mice and age-matched CBA-057Bl/6 F1 hybrid (CBF1) mice were obtained from Ace Laboratories (Boyertown, Pa.), Ace Laboratories and Jackson Laboratories (Bar Harbor, Me.), respectively. Founder Tg197 mice for the transgenic colony was obtained from G. Kollias. The breeding stock was maintained as homozygotes and received weekly injections of murine anti-human TNFa antibodies (10 mg/kg intraperitoneally) to control their arthritis. For these experiments, homozygous Tg197 males were bred to CBA females. CBF1 mice were used because their disease progresses more slowly than that seen in homozygous Tg197 mice. The mice were group housed (4 per cage) in filter-topped plastic shoebox style cages. The animals were individually identified with ear tags, placed at least a week prior to the start of the study. Food and water were available ad libitum and the room had a 12 hr light/dark cycle. All mice were maintained in the pathogen-free vivarium at Centocor R&D, Inc., Radnor Pa. The Institutional Animal Care and Use Committee at Centocor approved all associated procedures.

CNTO 530, recombinant human erythropoietin (epoetin-α, OrthoBiotech, Raritian N.J.), darbepoetin (Amgen, Thousand Oaks, Calif.), and PBS. Doses were expressed as mg/kg or UT-7 Units/kg (U/kg).

To comparative the pharmacodynamics of epoetin-α in C57Bl/6 and Tg197 mice, study animals were assigned to groups as shown in Table 1. On Day 0 the mice received a weight-adjusted, subcutaneous injection of test article (freshly diluted from stock) or PBS. Mice were euthanized by CO₂ asphyxiation and blood was collected for each sample.

TABLE 1

| | | Number of | | Dose | Approximate | Blood Sampling Time |
|---|---|---|---|---|---|---|
| Group | Strain | mice/group | Treatment | (mg/kg) | Dose (U/kg) | after dosing (days) |
| 1 | C57Bl/6 | 4 | PBS | 0 | 0 | 4, 7, 15, and 21 |
| 2 | C57Bl/6 | 4 | epoetin-α | 0.03 | 3,000 | 4, 7, 15, and 21 |
| 3 | C57Bl/6 | 4 | epoetin-α | 0.3 | 30,000 | 4, 7, 15, and 21 |
| 4 | Tg197 | 8-11 | PBS | 0 | 0 | 4, 8, 15 and 23 |
| 5 | Tg197 | 4-5 | epoetin-α | 0.03 | 3,000 | 4, 8, 15 and 23 |
| 6 | Tg197 | 4 | epoetin-α | 0.3 | 30,000 | 4, 8, 15 and 23 |

Comparative pharmacodynamics of CNTO 530, epoetin-α and darbepoetin in Tg197 mice: Study animals were assigned to groups as shown in Table 2. On Day 0 the mice received a weight-adjusted, subcutaneous injection of test article (freshly diluted from stock) or PBS. Groups of mice were euthanized by CO2 asphyxiation and blood/bone marrow collected.

TABLE 2

| | Number of | | Dose | Approximate | Blood Sampling Time |
|---|---|---|---|---|---|
| Group | mice/group | Treatment | (mg/kg) | Dose (U/kg) | after dosing (days) |
| 1 | 9-20 | PBS | 0 | 0 | 3-4, 8-10, 15, 21-23, 28 |
| 2 | 4-5 | epoetin-α | 0.3 | 30,000 | 4, 8, 15, 23 |
| 3 | 3-4 | darbepoetin | 0.1 | 30,000 | 4, 8, 15, 22 or 28 |
| 4 | 4 | CNTO 530 | 1.0 | 30,000 | 3, 8, 15, 21 or 28 |

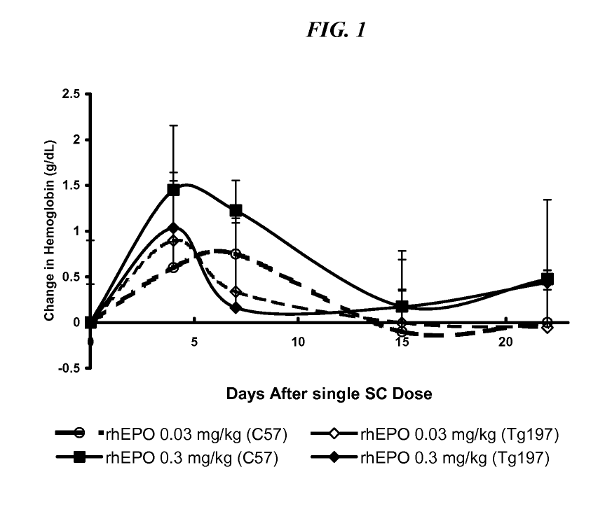

Hematology: Blood samples from mice in the model characterization and pharmacodynamic studies were collected at the times indicated in Tables 1 and 2. Blood was collected from mice anesthetized with a CO2 mixture via open chest cardiac puncture into commercially prepared EDTA coated microtubes. Blood analyses were performed on whole blood using the ADVIA 120 blood analyzer (Bayer Diagnostics, Tarrytown, N.Y.). Data are expressed as group mean±standard deviation.
Results: Data are expressed as the group mean and standard deviation or as group mean change from control. Mean values for the PBS control mice were used as the Day 1 baseline. For graphing, the nominal day may differ from the actual day of blood sampling by 1 day.
Mean (±standard deviation) hematological data for 30 CBF1 and 50 Tg197 PBS control mice (9 to 13 weeks old) are shown in Table 3. Tg197 mice showed slightly decreased Hgb (1 g/dL) and Hct (2%) compared to age matched CBF1 mice. Based on the normal reticulocyte and red blood cell counts and MCV and a slightly decreased MCH, Tg197 mice can be described as presenting with a mild, compensated, normocytic, hypochromic anemia.
Results: The results of a comparison of the pharmacodynamics of epoetin-α in C57Bl/6 and Tg197 mice are shown in FIG. 1. In C57Bl/6 mice, epoetin-α caused a dose-dependent increase in hemoglobin. In contrast, in Tg197 mice, there was little dose response between 0.03 and 0.3 mg/kg. Moreover, the Hgb response to 0.3 mg/kg epoetin-α in Tg197 mice was short-lived (returning to base line by Day 8) and blunted (˜1 g/dL) compared to the approximate 1.5 g/dL increase in Hgb that did not return to baseline until Day 15 seen in C57Bl/6 mice.

TABLE 3

| | Normal (CBF₁) | Arthritic (Tg197) |
|---|---|---|
| Parameter | 9-14 Week | 9-14 week |
| #Retic (×10\hat{ }9/L) | 243 ± 57 | 267 ± 76 |
| RBC (×10\hat{ }6/uL) | 9.80 ± 0.4 | 9.70 ± 0.6 |
| MCV (fL) | 53.8 ± 0.8 | 52.2 ± 2.2 |
| MCH (pg) | 16.3 ± 0.9 | 15.2 ± 0.9 |
| HGB (g/dL) | 15.9 ± 0.3 | 14.8 ± 1.0 |
| HCT (%) | 52.5 ± 2.1 | 50.7 ± 3.3 |

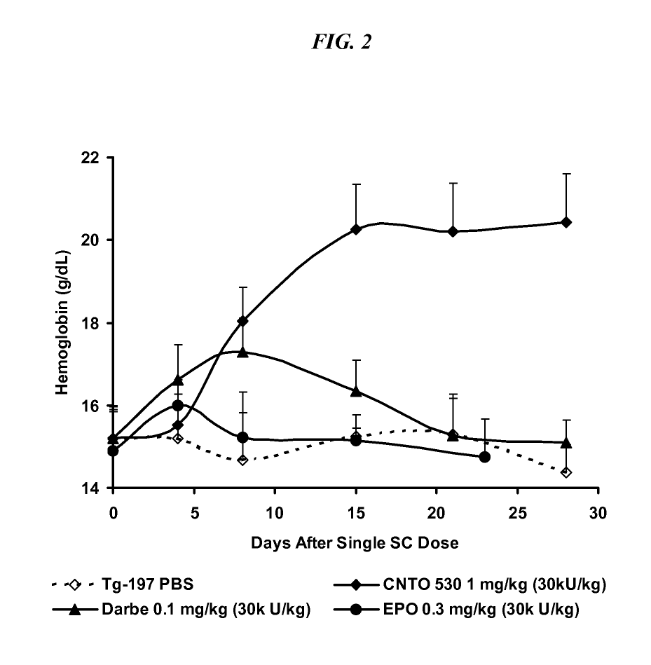

The results of the pharmacodynamic study in Tg197 mice are summarized in FIG. 2. To allow a direct comparison between CNTO 530, epoetin-α and darbepoetin, doses were converted to UT-7 units/kg. CNTO 530 caused a stronger and longer-lived response in Hgb than either epoetin-α or darbepoetin in the model of EPO resistant anemia of chronic disease.

### Example 2

### EPO Resistance 1N Stem Cell Factor Receptor Deficiency

Mice deficient in c-kit the receptor for stem cell factor were used to demonstrate the effect of adjunctive receptors in the hematopoietic process.
Materials and Methods. Male and female WBB6F1/J-KitW/KitW-v (black eyed, white coat, affected; Related genotype ala KitW/KitW-v) (W/Wv) mice were obtained from Jackson Laboratories, Bar Harbor, Me. at 5 to 7 weeks of age. These mice are deficient in c-kit the receptor for SCF. The mice were group housed in filter topped plastic shoe-box style cages. CNTO 530 (30 UT-7 Units/ug) and epoetin-α (120 UT-7 Units/ug) were tested and PBS pH 7.4 was used as the control article.
Study Design: On Day 1 mice received a weight-adjusted, subcutaneous (s.c.) dose of epoetin-α, CNTO 530 or PBS (10 mL/kg) according to Table 4. Three mice/sex were bled per group at each designated time point according to Table 4. On Days 4, 9, 14, and 28 (males) or 30 (females), mice were anesthetized with CO2 and blood samples (−0.4 mL) taken via open chest cardiac puncture for hematology. Blood was collected directly into tubes prepared with EDTA and mixed thoroughly for approximately 10 seconds and placed on a rocker to prevent coagulation. Whole blood was be analyzed using an ADVIA 120 hematology analyzer.

TABLE 4

| | Mouse | | Dose | Dose |
|---|---|---|---|---|
| Group | Strain | Treatment | (mg/kg) | (UT-7 Units/kg) |
| 1 | Ala +/+ | PBS | 0 | 0 |
| 2 | Ala +/+ | epoetin-α | 0.1 | 12,000 |
| 3 | Ala +/+ | CNTO 530 | 0.4 | 12,000 |
| 4 | W/Wv | PBS | 0 | 0 |
| 5 | W/Wv | epoetin-α | 0.1 | 12,000 |
| 6 | W/Wv | CNTO 530 | 0.4 | 12,000 |

Results. Data are expressed as the group mean and standard deviation or as group mean change from control. Mean values for the PBS control mice were used as the Day 1 baseline. For graphing, the nominal day may differ from the actual day of blood sampling by 1 day.

Hematologic analysis of ala +/+ and W/Wv mice that received PBS revealed that the W/Wv mice had a mild, macrocytic, normochromic anemia (Table 5). Although the % reticulocytes and the number of high RNA content reticulocytes in the W/Wv mice was approximately 2 fold higher than the ala +/+ control mice, the absolute number of reticulocytes was similar. Taken together, the increased MCV, MCH and high RNA reticulocyte count and normal total reticulocyte count suggest that in addition to a deficiency at the level of the stem cell, these mice also have a defect in the later stages of RBC maturation.

TABLE 5

| Mice | ala +/+ | W/Wv |
|---|---|---|
| HGB (g/dL) | 15.5 ± 0.5 | 12.1 ± 1.2 |
| HCT (%) | 52.5 ± 1.7 | 40.9 ± 3.7 |
| RBC (×10⁶/uL) | 10.2 ± 0.4 | 6.1 ± 0.7 |
| % Retic | 3.3 ± 0.6 | 6 ± 3 |
| #Retic (10⁹/L) | 331.8 ± 62.2 | 329 ± 102 |
| High RNA Retic (10⁹/L) | 74.3 ± 30.2 | 150.1 ± 77.7 |
| RDW (%) | 12.0 ± 0.8 | 16.2 ± 1.9 |
| retic_MCV (fL) | 61.3 ± 1.3 | 81.2 ± 2.5 |
| MCH (pg) | 15.1 ± 0.4 | 19.8 ± 0.6 |
| MCHC (g/dL) | 29.5 ± 0.8 | 29.5 ± 0.8 |

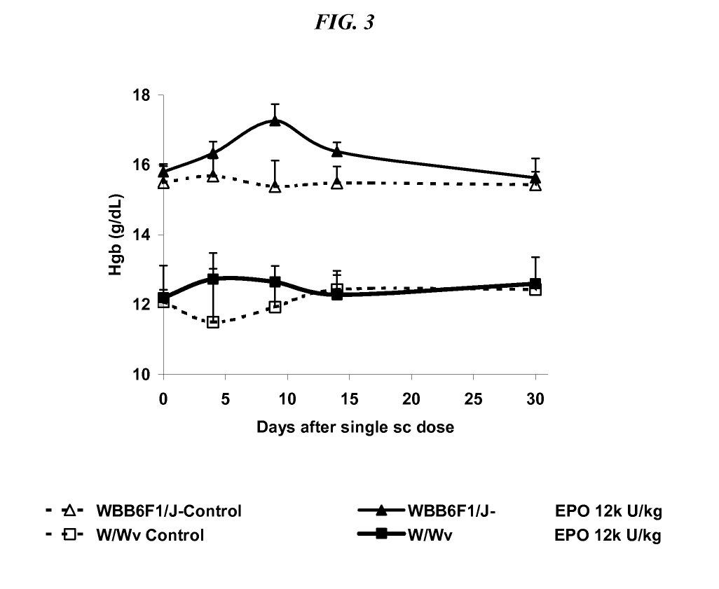

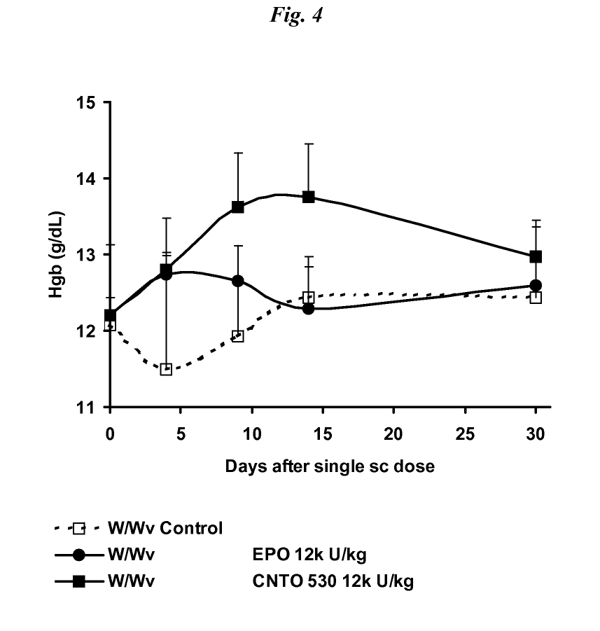

The data supporting that c-kit deficient mice are hyporesponsive to epoetin-α are shown in FIG. 3. In contrast to an increase of approximately 2 g/dL in Hgb following a single subcutaneous dose of epoetin-α in the normal ala +/+ littermates, there was less than a 1 g/dL increase in Hgb in the W/Wv mice. The comparative hemoglobin response of W/Wv mice to ESAs is shown in FIG. 4. A single subcutaneous dose of CNTO 530 of 12,000 U/kg dose caused a long-lived, approximately 2 g/dL increase in hemoglobin compared to the less than 1 g/dL, short lived increase in Hgb observed in response to epoetin-α. Thus, CNTO 530 caused a stronger and longer-lived response in Hgb than rHuEPO in this EPO resistant model of anemia secondary to a stem cell defect.

### Example 3

### An EPO Resistant Model of B-Thalassemia

Th3+/C57BL/6 mice are heterozygous for a deletion of both the b1 and b2 globin gene (Yang et al. 1995. Proc Nat Acad Sci, USA, 92:11608-11612) and are therefore useful in modeling the dysregulation of hemoglobin synthesis (hemoglobinopathy) that leads the anemia associated with beta-thalassemia.

Materials and Methods. Male and female Th3+/C57BL/6 (heterozygous) mice maintained in a pathogen-free vivarium. Founder Th3+/C57BL/6 mice for the colony was obtained from the Univ Penn. The breeding stock was maintained as heterozygotes. Th3+/C57BL/6 were selected for the pharmacodynamics study based on a pale visual appearance and splenomegaly. The selection strategy was validated in a pilot study (see below). CNTO 530, recombinant human erythropoietin (epoetin-α) (OrthoBiotech, Raritian N.J.), darbepoetin (ARANESP™, Amgen, Thousand Oaks, Calif.) were tested. Doses were expressed as mg/kg or UT-7 Units/kg (U/kg).
Pilot study design: Seven Th3+/C57BL/6 and 7 normal littermates were anesthetized with CO₂ and blood samples (0.4 mL) taken via open chest cardiac puncture for hematology. Blood was collected directly into tubes prepared with EDTA and mixed thoroughly for approximately 10 seconds and placed on a rocker to prevent coagulation. Whole blood was analyzed using an ADVIA 120 hematology analyzer.
Study of comparative pharmacodynamics of CNTO 530, epoetin-α and darbepoetin in Th3+/C57BL/6 mice: On Day 0 the mice received a weight-adjusted, subcutaneous injection of test article (freshly diluted from stock) or PBS. On Day 1, groups of 8 mice received a weight-adjusted, subcutaneous (s.c.) dose of rhEPO, CNTO 530, epoetin-α or darbepoetin CNTO 530 formulation buffer (10 mL/kg) according to Table 6.

TABLE 6

| | | | Blood Sample Collection |
|---|---|---|---|
| Group | Treatment (Day 1) | Test Article Dose (s.c.) | (2/sex/time point) |
| 1 | Control | 0 | Days 4, 8, 10, 15, 22 |
| 2 | CNTO 530 | 0.3 mg/kg (~10,000 U/kg) | Days 4, 8, 10, 15, 22 |
| 3 | epoetin-α | 0.1 mg/kg (~10,000 U/kg) | Days 4, 8, 10, 15, 22 |
| 4 | Darbepoetin | 0.03 mg/kg (~10,000 U/kg) | Days 4, 8, 10, 15, 22 |

Hematology: Blood samples from mice in the model characterization pilot study were collected at a single time point. Blood samples from mice in the pharmacodynamic studies were collected at the times indicated in Table 6. Blood was collected from mice anesthetized with a CO2 mixture via open chest cardiac puncture into commercially prepared EDTA coated microtubes. Blood analyses were performed on whole blood using the ADVIA 120 blood analyzer (Bayer Diagnostics, Tarrytown, N.Y.). Data are expressed as group mean±standard deviation. Data are expressed as the group mean and standard deviation or as group mean change from control. Mean values for the PBS control mice were used as the Day 1 baseline. For graphing, the nominal day may differ from the actual day of blood sampling by 1 day.

### Results

The results of the hematologic analysis of C57BL/6 and Th3+/C57BL/6 littermates is shown in Table 7. Th3+/C57BL/6 mice showed decreased RBC, Hgb and Hct (2%) compared to age matched C57BL/6 mice, confirming their assignment as Th3+/C57BL/6. Based on the markedly increased reticulocyte counts, % reticuolocytes and high RNA reticulocytes count it is evident that these mice are trying to correct their anemia. That this attempt is ineffective is reflected by the smaller mean cell volume (MCV), the increased red cell distribution width (RDW) and decreased cellular hemoglobin indices. Thus, the Th3+/C57BL/6 mice can be described as presenting with a marked, microcytic, hypochromic, and regenerative anemia.

TABLE 7

| | C57BL/6 | Th3⁺/C57BL/6 |
|---|---|---|
| HGB (g/dL) | 15.6 ± 0.4 | 9.1 ± 0.6 |
| HCT (%) | 50.8 ± 1.5 | 35.2 ± 2.1 |
| RBC (10⁶/uL) | 10.4 ± 0.4 | 8.5 ± 0.4 |
| % Retic | 2 ± 0.3 | 23.6 ± 1.7 |
| #Retic (10⁹/L) | 203 ± 23 | 1961 ± 415 |
| High RNA Retic (10⁹/L) | 34.7 ± 13.2 | 940.7 ± 68.5 |
| RDW (%) | 13.5 ± 0.8 | 37.1 ± 1.2 |
| MCV (fL) | 48.9 ± 1.0 | 39.9 ± 0.9 |
| MCH (pg) | 15.0 ± 0.4 | 10.3 ± 0.4 |
| MCHC (g/dL) | 30.7 ± 0.4 | 25.9 ± 0.5 |

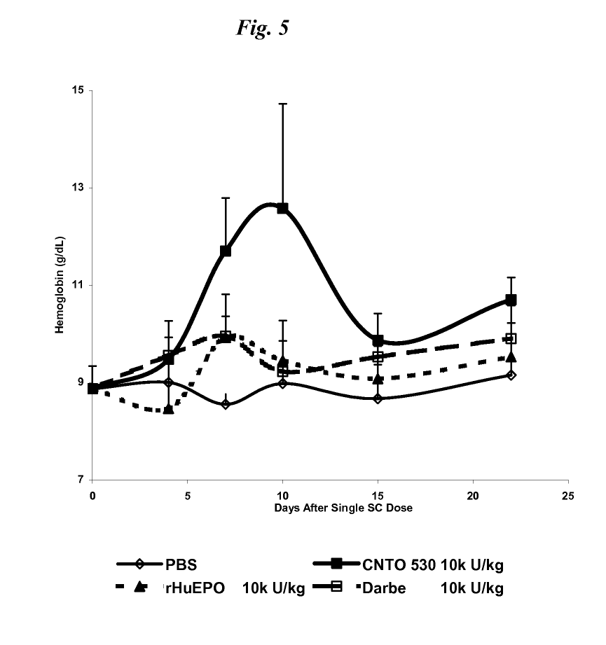

Pharmacodynamics of CNTO 530, epoetin-α and darbepoetin in Th3+/C57BL/6: The results of are shown graphically in FIG. 5. To allow a direct comparison between CNTO 530, epoetin-α and darbepoetin, doses are expressed as UT-7 units/kg. A single subcutaneous dose of CNTO 530 of 10,000 U/kg dose caused a long-lived, approximately 4 g/dL increase in hemoglobin compared to the less than 1 g/dL, short lived increase in Hgb observed in response to epoetin-α or darbepoetin. Thus, CNTO 530 caused a stronger and longer-lived response in Hgb than epoetin-α in this EPO hyporesponsive model of b-thalassemia.

### Example 4

### CNTO530 in an Animal Model of Sickle Cell Disease

In sickle cell disease, anemia results from defective red cell production or shortened red blood cell life span. A possible method for amelioration or prevention of damage to red cells caused by the sickle cell hemoglobin is for fetal hemoglobin to replace or represent at least a portion of the red cell hemoglobin. The ability of CNTO530 to stimulate fetal hemoglobin synthesis was examined

### Materials and Methods

Ten-Sixteen week old Hba Hbatm1P tm1Paz az Hb Hbbtm1T tm1Tow Tg(HBA-HBB (HBBs)4) 41P 1 Paz/Jaz/mice were obtained from Ace Laboratories (Boyertown, Pa.). Founder mice for the transgenic colony were obtained from Jackson Laboratories (Bar Harbor Me.). As originally described by Pászty et al (Pászty et al. 1997 Science 278:876-878) the gene for murine alpha and beta globin are disrupted (knocked out) and are transgenic for human alpha, beta (sickle) and gamma globin genes. Thus, they express exclusively human hemoglobin A (HbAsickle) (or sickle hemoglobin, HbS) and can also express human fetal hemoglobin (HbF).

The mice were group-housed in filter-topped plastic shoebox style cages. A total of 7 mice were used. The experiments were conducted in three parts. On Day −7 the mice were anesthetized with a CO₂ mixture and bled with capillary tubes from the retro-orbital plexus into EDTA coated microtubes for evaluation of HbF (by ion exchange chromatography and electrophoresis) and for enumeration of % RBC containing HbF (by flow cytometry). On Day 1, the mice received a weight-adjusted, subcutaneous injection of CNTO 530 (freshly diluted from stock). Doses were expressed as mg/kg. On Day 9, blood was collected from mice anesthetized with a CO₂ mixture via open chest cardiac puncture into EDTA coated microtubes.

Hematology analyses were performed on whole blood from Day 9 using ADVIA 120 blood analyzer (Siemens Diagnostic Solutions, Tarrytown, N.Y.). Total Hgb values from the hematology analyzer and the results of a whole blood dilution series measured spectrophotometrically (OD 415) were used to calculate pre-dose total Hgb and change in total Hgb (See below).

Ion exchange chromatorgraphic analysis of HbF was performed after the method of Morin and Barton (Morin and Barton 1987). Briefly, 50 μL fresh whole blood was lysed in 200 μL distilled H₂O containing 0.1% Triton-X 100 and 200 mM KCN, frozen and stored at −70° C. On the day of analysis, the pre- and post-dose samples were thawed and 1 mL adsorbtion buffer was added. The adsorbtion buffer contained 200 mM Bis-Tris acetate (pH 4.5), 200 mM KCN and a trace amount of trichlorobutanol as a preservative. One cm disposable mini-columns were packed with 3 mL of a slurry of Sephadex CM-50 (10 g/400 mL in adsorbtion buffer). The column was allowed to drain under minimal vacuum, the packing covered with a glass frit and washed with adsorbtion buffer.

One 1 ml of lysed whole blood layered on the packing. Pre- and post-dose samples were run side by side. The column was washed with 2, 1 mL aliquots of adsorbtion buffer to remove unbound hemoglobin. The column was eluted with 2 mL aliquots of elution buffer and 2 mL fractions were collected under gravity. (The elution buffer contained 100 mM Bis-Tris acetate (pH 6), 4.8 g/L magnesium acetate, 200 mM KCN, and a trace amount of trichlorobutanol as a preservative.)

Aliquots of the collected fractions (0.33 mL) were transferred to a 96 well plate and the OD 415 (soret peak for Hgb) read on a Molecular Devices SpectraMax 340PC (Sunnyvale, Calif.). Pre- and post-dose HbF ratio was calculated using the peak value (Fraction 4) for each animal. Data are expressed as mean±standard deviation. Statistical significance was determined by t-test. P values <0.05 were accepted as significant.

To calculate the total hemoglobin concentration, 250 μL of the remaining whole blood lysate was diluted to 2 mL and serial 2 fold dilutions prepared in adsorbtion buffer. Aliquots of these dilutions (0.33 mL) were transferred to a 96 well plate and the OD at 415 nm recorded. These values were used with the total Hgb measured by the hematology analyzer to calculate the predose value and drug induced change in total Hgb. Data are expressed as mean±standard deviation. Statistical significance was determined by t-test. P values <0.05 were accepted as significant.

Flow cytometric analysis of RBC and reticulocytes expressing fetal hemoglobin was performed after the method of after the method of B Davis and K Davis (Current Protocols in Cytometry, 2004). Briefly, about 25×10⁶ RBC were fixed with 1 mL cold 0.05% glutaraldehyde in phosphate buffered saline (PBS) for 10 minutes. The cells were washed with 2 mL 0.1% bovine serum albumin (BSA) 0.1% sodium azide in PBS (BSA-PBS) and permeabilized in 500 μL 0.1% Triton-X 100 (in BSA-PBS) for 3-5 minutes, washed and resuspended in 500 μL BSA-PBS. Ten μL aliquots were stained with anti-HbF antibodies (5 μL in 80 μL BSA-PBS) in a 96 well round bottom plate for 15 minutes. (Murine monoclonal anti-human HbF, clone HbF−1 (12) Cy5 (TRI-COLOR®, TC) (Catalog No. HFH-06, Invitrogen The cells were washed and resuspended in 200 μL thiazole orange (Retic-Count™Reticulocyte Reagent System, Becton Dickinson Biosciences, San Jose, Calif., Catalog No. 349204) for 15-30 minutes. Staining was controlled with a Fetal Hemoglobin Control Kit (Fetaltrol), Invitrogen, Catalog No. FH102 and BD Retic-Count™ Control Kit (Tri-Level Control), Catalog No. 340999.

Data were acquired on a Becton Dickinson FACSCalibur. Monodisperse cells were gated on the basis of forward and side scatter. Cells stained with HbF−1 were counted as HbF+ and cells stained with thiazole orange were counted as reticulocytes.

Data are expressed as mean±standard deviation for % HbF+ reticulocytes and % HbF+ total RBC. Because the data were not normally distributed, they were log transformed for statistical analysis by t-test. P values <0.05 were accepted as significant.

Electrophoretic analysis of hemoglobin was performed with a QuickGel® acid hemoglobin kit (catalogue No. 3519) a QuickGel® chamber (catalogue No. 1284) and a Titan Plus power supply (catalogue No. 1504) (Helena Laboratories, Beaumont, Tex.). All reagents were used as supplied according to the manufacturer's instructions except that the gels were loaded with 34 uL of lysates and run for 23 minutes at 140 volts. AFSC Hemo Control (catalogue No. 5331) was used as a control.

### Results

The effects of CNTO 530 on total Hgb are shown in Table 8. Nine days after receiving a single sc dose of CNTO 530 (0.3 mg/kg) there was a statistically significant (5.8 g/dL) increase in total Hgb.

TABLE 8

| | | Post-Dose: | | Post-Dose |
|---|---|---|---|---|
| | Total Hgb | Pre-Dose | Total Hgb | Increase |
| | Day 9 Post- | Ratio (Mean | Day-7 Pre- | in Total |
| Animal | Dose (g/dL) | OD 415) | Dose (g/dL) | Hgb (g/dL) |
| P-2008-170-1 | 12.9 | 1.6 | 8.1 | 4.8 |
| P-2008-170-2 | 12.0 | 1.9 | 6.2 | 5.8 |
| P-2008-192-1 | 7.1 | 0.9 | 7.8 | −0.7 |
| P-2008-239-1 | 16.1 | 1.6 | 10.2 | 5.9 |
| P-2008-239-2 | 17.8 | 1.8 | 9.9 | 7.9 |
| P-2008-240-1 | 18.6 | 2.5 | 7.5 | 11.1 |
| P-2008-240-2 | No sample | 2.0 | — | — |
| Mean ± SD | 14.1 ± 4.3* | 1.8 ± 0.5 | 8.3 ± 1.5 | 5.8 ± 3.9 |

*Statistically greater than pre-dose (P = 0.011, t-test)

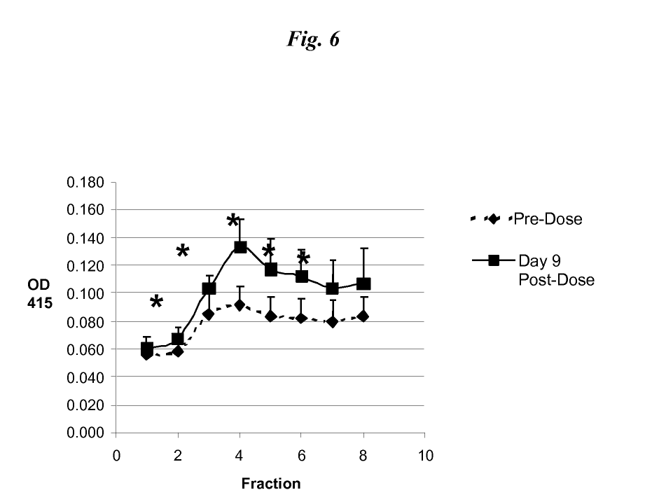

Effects of CNTO 530 on HbF (Ion Exchange Chromatography): The results of the ion exchange chromatography are shown in FIG. 6 and Table 9. Nine days after receiving a single sc dose of CNTO 530 (0.3 mg/kg) there was a statistically significant increase in HgF. There was no significant difference between the fold increase in total Hgb and fold increase HbF (t-test).

TABLE 9

| | Fold Increase Total Hgb | Fold Increase HbF |
|---|---|---|
| Animal | (OD 415) | (OD 415) |
| P-2008-170-1 | 1.6 | 1.4 |
| P-2008-170-2 | 1.9 | 1.7 |
| P-2008-192-1 | 0.9 | 1.2 |
| P-2008-239-1 | 1.6 | 1.3 |
| P-2008-239-2 | 1.8 | 1.6 |
| P-2008-240-1 | 2.5 | 1.6 |
| P-2008-240-2 | 2.0 | 1.5 |
| Mean ± SD | 1.8 ± 0.5 | 1.5 ± 0.2 |

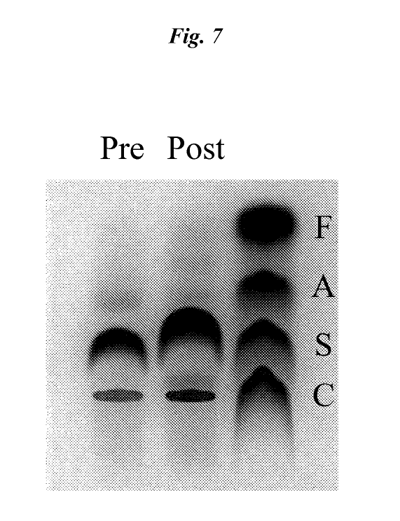

The results of the Hgb electrophoresis are shown in FIG. 7 and Table 10. Nine days after receiving a single sc dose of CNTO 530 (0.3 mg/kg), although the HbF bands were too weak to quantitate, there was a discernable increase in the HbS and HbF bands for all 7 mice.

TABLE 10

| | HbS | | HbF | |
|---|---|---|---|---|
| Animal | Pre-Dose | Post-Dose | Pre-Dose | Post-Dose |
| P-2008-170-1 | +++ | ++++ | − | + |
| P-2008-170-2 | +++ | ++++ | − | + |
| P-2008-192-1 | +++ | ++++ | − | + |
| P-2008-239-1 | ++ | ++++ | − | +/− |
| P-2008-239-2 | +++ | ++++ | +/− | + |
| P-2008-240-1 | ++ | +++ | − | +/− |
| P-2008-240-2 | +++ | ++++ | +/− | + |

Effects of CNTO 530 on HbF+ Cells: The results of the flow cytometric analysis of HbF+ cells are shown Tables 11 and 12. Nine days after receiving a single sc dose of CNTO 530 (0.3 mg/kg) there was a trend toward an increase in % HbF+reticulocytes (4.5 fold) and a statistically significant increase in total % HbF+ cells (reticulocytes and RBC) (3.7 fold).

TABLE 11
Effects of CNTO 530 on HbF + Reticulocytes

| | % HbF + | % HbF + | Fold Increase % |
|---|---|---|---|
| | Reticulocytes | Reticulocytes | HbF + |
| Animal | Pre-Dose | Post-Dose | Reticulocytes |
| P-2008-170-1 | 2.7 | 1.9 | 0.7 |
| P-2008-170-2 | 2.5 | 1.6 | 0.6 |
| P-2008-192-1 | 7.8 | 22.4 | 2.9 |
| P-2008-239-1 | 0.3 | 2.1 | 6.3 |
| P-2008-239-2 | 0.3 | 1.3 | 3.8 |
| P-2008-240-1 | 0.6 | 2.6 | 4.6 |
| P-2008-240-2 | 0.3 | 4.2 | 12.7 |
| Mean ± SD | 0.4 ± 0.1 | 2.5 ± 1.2 | 4.5 ± 4.1 |

* Statistically greater than pre-dose (P = 0.011, t-test)

TABLE 12

| | Total % HbF + | Total % HbF + | Fold Increase % |
|---|---|---|---|
| Animal | Cells Pre-Dose | Cells Post-Dose | Total HbF + Cells |
| P-2008-170-1 | 3.0 | 3.8 | 1.3 |
| P-2008-170-2 | 3.1 | 2.8 | 0.9 |
| P-2008-192-1 | 8.5 | 28.0 | 3.3 |
| P-2008-239-1 | 1.5 | 4.0 | 2.7 |
| P-2008-239-2 | 0.9 | 2.9 | 3.1 |
| P-2008-240-1 | 0.9 | 3.7 | 4.2 |
| P-2008-240-2 | 0.7 | 7.1 | 10.4 |
| Mean ± SD | 1.0 ± 0.3 | 4.4 ± 1.8* | 3.7 ± 3.2 |

*Statistically greater than pre-dose (P = 0.044, t-test)

### Summary

A single sc dose of CNTO 530 increases expression of fetal hemoglobin (HbF) in a murine model of sickle cell anemia 9 days after dosing. Increased expression of HbF is associated with an increase in organ function in sickle cell mice (Fabry et al. 2001 Blood 97:410-418) and a decreased incidence of sickle cell crisis (Moore et al. 2000 Hematol 64:26-31). Therefore, long-term treatment with CNTO 530 could be considered to improve the anemia of sickle cell disease and decrease the incidence of sickle cell crisis.

### REFERENCES

Foote, Mary Ann; Colowick, Alan; Goodkin, David A. Amgen Inc., Thousand Oaks, Calif., USA. Pharmacologic and cytokine treatment of commonly encountered anemias. Cytokines, Cellular & Molecular Therapy (2002), 7(2), 49-59. Publisher: Martin Dunitz Ltd.
Q. Fan, K. Leuther, C. Holmes, K. Fong, J. Zhang, S. Velkovska, M. Chen, R. Mortensen, K. Leu, J. Green. 2006. Preclinical evaluation of Hematide, a novel erythropoiesis stimulating agent, for the treatment of anemia. Experimental Hematology, Volume 34, Issue 10, Pages 1303-1311
Functional mimicry of a protein hormone by a peptide agonist: the EPO receptor complex at 2.8 ANG. Livnah, Oded; Stura, Enrico A.; Johnson, Dana L.; Middleton, Steven A.; Mulcahy, Linda S.; Wrighton, Nicholas C.; Dower, William J.; Jolliffe, Linda K.; Wilson, Ian A. Dep. of Molecular Biology, The Scripps Research Inst., La Jolla, Calif., USA. Science (Washington, D.C.) (1996), 273(5274), 464-471.
Yang B, Suzanne K, Lewis J, Detloff P J, Maeda N, and Smithies O, A mouse model for β-thalassemia. Proc Nat Acad Sci, USA, Vol 92: 11608-11612, December 1995.
T. Kitamura, A. Tojo, T. Kuwaki, S. Chiba, K. Miyazono, A. Urabe and F. Takaku, Identification and analysis of human erythropoietin receptors on a factor dependent cell line, TF-1, Blood 73: 375-380 (1989).
Dufour C, Corcione A, Svahn J, et al. TNF-alpha and IFN-gamma are overexpressed in the bone marrow of Fanconi anemia patients and TNF-alpha suppresses erythropoiesis in vitro. Blood 2003; 102:2053-2059.
Munugalavadla V, Dore L C, Tan B L, et al. Repression of c-kit and its downstream substrates by GATA-1 inhibits cell proliferation during erythroid maturation. Mol Cell Biol 2005; 25:6747-6759.
Francesco Locatelli, Bruno Reigner. C.E.R.A.: pharmacodynamics, pharmacokinetics and efficacy in patients with chronic kidney disease. Expert Opinion on Investigational Drugs, October 2007, Vol. 16, No. 10, Pages 1649-1661(doi: 1 0.1517/13543784.16.10.1649)
Woodburn K W, Fan Q, Leuther K K, et al. Preclinical evaluation of Hematide[TM], a novel erythropoietic receptor agonist for the treatment of anemia caused by kidney disease. Blood. 2004; 104:2904.
Stead R B, Lambert J, Wessels D, et al. Evaluation of the safety and pharmacodynamics of Hematide, a novel erythropoietic agent, in a phase 1, double-blind, placebo-controlled, dose-escalation study in healthy volunteers. Blood. 2006; 108:1830-1834.
Duliege A, Macdougall I, Duncan N, et al. Hematide, a synthetic peptide-based erythropoiesis stimulating agent (ESA), demonstrates erythropoietic activity in a phase 2 single dose, dose escalating study in patients with chronic kidney disease (CKD). Blood. ASH Annual Meeting Abstracts. 2005; 106:Abstract 3532.
FibroGen Reports Phase 1 Results of Investigational Oral Anemia Therapy, FG-2216, and New Data from Phase 2A Study in Anemic Chronic Kidney Disease Patients. Available at: www.fibrogen.com/news/fg20041206.html. Accessed May 9, 2007.

What is claimed:

1. A method for treating a patient having a disorder characterized by a low blood hemoglobin level or a low level of red blood cells in the blood characterized as anemia caused by a hemoglobinopathy or myelodysplasia, which method comprises contacting the hematopoietic tissue of the patient with a therapeutically effective amount of the compound comprising dimeric polypeptides in which each polypeptide comprises an erythropoietin mimetic peptide (EMP) and a human immunoglobulin domain, wherein the dimeric polypeptide composition is capable of causing erythropoietin-dependent cells to proliferate.

2. A method according to claim 1 wherein the cause of the anemia is selected from the group consisting of end stage renal failure or dialysis; anemia associated with AIDS, auto immune disease; beta-thalassemia; sickle cell disease; cystic fibrosis; anemia associated with chronic inflammatory disease; anemia of aging; and neoplastic disease.

3. The method according to claim 1, wherein the EMP composition treats an anemia derived from a condition characterized by a defect or deficiency in stem cell factor receptors.

4. The method according to claim 1, in which said EPO-mimetic peptide composition treats a hemoglobinopathy selected from the group consisting of sickle cell anemia, thalassemia, hemoglobinopathy which is acquired, or hemoglobinopathy related to structural variants of human hemoglobin.

5. The method according to claim 1, wherein the EPO-mimetic peptide composition comprises homodimerized disulfide linked polypeptides of either SEQ ID NO: 2 or 3.

6. A method of claim 1 to 5, wherein the therapeutically effective amount of the dimeric EMP-polypeptide composition is calculated relative to rhEPO using a UT7 cell proliferation assay.

7. The method of claim 1, wherein the anemia is caused by bone marrow failure and the hematopoietic tissue of the patient is bone marrow which has been contacted with the dimeric EMP-polypeptide composition ex vivo.

8. The method of claim 7, wherein the bone marrow tissue is cultured ex vivo prior to returning the tissue to the patient.

9. The method of claim 7 or 8, wherein the bone marrow tissue is contacted by additional hematopoiesis stimulating factors including at least one of SCF, G-CSF, IL-3, GM-CSF, IL-6 or IL-11.

10. The method of any of claims 1 to 9, wherein the patient is additionally administered a source of iron.
